(19) United States
(12) Patent Application Publication
(10) Pub. No.: US 2010/0210836 A1
(43) Pub. Date: Aug. 19, 2010

(54) SMALL MOLECULE INHIBITORS OF LYMPHOID TYROSINE PHOSPHATASE

(75) Inventors: Nunzio BOTTINI, San Diego, CA (US); Nouri Neamati, Fullerton, CA (US)

Correspondence Address: Hogan Lovells US LLP, 1999 AVENUE OF THE STARS, SUITE 1400, LOS ANGELES, CA, 90067, US

(73) Assignee: University of Southern California, Los Angeles, CA (US)

(21) Appl. No.: 12/705,198

(22) Filed: Feb. 12, 2010

Related U.S. Application Data
(60) Provisional application No. 61/152,612, filed on Feb. 13, 2009.

(51) Int. Cl. C07D 401/14; C07D 413/14

(52) U.S. Cl. 540/597; 544/128; 544/363

(57) ABSTRACT

The present invention relates to lymphoid tyrosine phosphatase inhibitors and autoimmunity. More specifically, the invention relates to the identification of lymphoid tyrosine phosphatase inhibitors and the analysis of the structure-activity relationship.

The present application claims the benefit of the filing date of U.S. Provisional Application No. 61/152,612 filed Feb. 13, 2009, the disclosure of which is incorporated herein by reference in its entirety.

### STATEMENT REGARDING FEDERALLY SPONSORED RESEARCH OR DEVELOPMENT

This invention was made with government support under Contract No. 1R21NS056945-01 awarded by the National Institutes of Health. The government has certain rights in the invention.

### FIELD OF THE INVENTION

The present invention relates in general to autoimmunity. More specifically, the invention provides lymphoid tyrosine phosphatase inhibitors that may be useful as a therapeutic for autoimmunity.

### BACKGROUND OF THE INVENTION

Autoimmune diseases are common diseases of complex etiology, in which a combination of genetic and environmental factors cause a loss of central and/or peripheral immune tolerance, leading to an attack against self-antigens (1,2). Autoimmunity includes a wide and heterogeneous range of pathological and clinical manifestation, but different autoimmune diseases can share similar pathogenic mechanisms leading to loss of immune tolerance (3). Among these “shared autoimmunity” mechanisms, signaling through the T cell receptor (TCR) plays an important role in two of the most important mechanisms of tolerance, the thymic selection, and the generation/function of regulatory T cells (2,4). Thus regulators of TCR signaling are currently important candidate genes and drug targets for human autoimmunity. Engagement of the TCR leads to a spatially and temporally regulated activation of protein tyrosine kinases (PTKs) and protein tyrosine phosphatases (PTPs), which leads to tyrosine phosphorylation and dephosphorylation of numerous intracellular proteins (5,6). Protein tyrosine phosphatases (PTPs) are very active mediators of TCR signaling and anomalies of expression and/or genetic polymorphisms of PTPs are associated with autoimmunity in mice and/or humans (710).

PTPs are also considered as promising drug targets in human autoimmunity, for example small molecule inhibitors of CD45 are currently sought for prevention and treatment of human diseases (8,11,12). A second PTP which recently emerged as a likely drug target for human autoimmunity is the lymphoid tyrosine phosphatase (LYP), which is encoded by the gene PTPN22, and belongs to a sub-family of PEST-enriched tyrosine phosphatases, including two additional enzymes, PTP-PEST (encoded by the PTPN12 gene), and BDP1 (encoded by the PTPN18 gene) (13,14). A missense polymorphism, C1858T (R620W) at the PTPN22 gene is an important risk factor in type 1 diabetes (T1D), rheumatoid arthritis (RS), Graves' disease, and other autoimmune diseases in multiple populations (10, 15-17). The association between the PTPN22 C1858T polymorphism and autoimmunity has been replicated in numerous populations, by case-control, family-based, and more recently genome-wide association studies (18,19). The polymorphism is primarily associated with T1D and RA (18), thus very likely to play a causal role in the diseases. Recent genome-wide scans also showed that with an odds ratio around 2, PTPN22 stands as the third most relevant gene in T1D in Caucasian populations, after HLA and INS, and the second most relevant gene in RA after HLA (20). LYP is expressed exclusively in hematopoietic cells and in T cells it is an important negative regulator of signal transduction through the T cell receptor (TCR) (21). The mechanism of regulation of signaling in T cells by LYP includes the formation of a complex between the phosphatase and the negative regulatory kinase Csk (22). The C1858T polymorphism results in the substitution of an Arg with a Trp in position 620 (R620W) of the protein, which is located in the LYP-Csk interaction motif and strongly reduces the affinity of LYP for Csk (16,23). LYP-W620 is a gain-of-function form of the enzyme, and carriers of this variant show reduced signal transduction through the TCR receptor (24). The mechanism of the gain-of-function phenotype and its relationship with Csk binding remains to be clarified. Reduced signaling in T cells is believed to play a role in the pathogenic mechanism of LYP-W620, by affecting either thymic selection, or the activity of regulatory T cells in the periphery (18).

Given the gain-of-function nature of the autoimmune-causing LYP-W620 variation, it has been proposed that specific small molecule inhibitors of LYP would be able to correct the signaling phenotype of LYP-W620 carriers, thus preventing or treating autoimmunity (24,25). Such anti-LYP therapy would effectively correct one of the biological mechanisms of disease, at least in subjects carrying the W620 variant. Besides the therapeutic relevance, specific LYP inhibitors will also be of invaluable help in understanding the mechanism of action of LYP by providing complementary information to genetic manipulations of the PTPN22 gene expression. In this study we set out to develop small-molecule inhibitors of LYP by a combination of chemical library screening, docking and validation of the leads obtained from screening in cells.

### SUMMARY OF THE INVENTION

This invention is based, at least in part, on the unexpected discovery that novel compounds described below can be used for treating all autoimmune diseases that are associated with PTPN22 genetic polymorphism, including: type 1 diabetes, rheumatoid arthritis, systemic lupus erythematosus, Graves' disease, Addison's disease, vitiligo, juvenile arthritis, Hashimoto thyroiditis, and other rarer diseases. The compounds can be injected or orally administered.

In one embodiment, the invention relates to a compound, or a pharmaceutically acceptable salt, solvate, or hydrate thereof, wherein the compound comprises a general formula 1 from the general class 1,

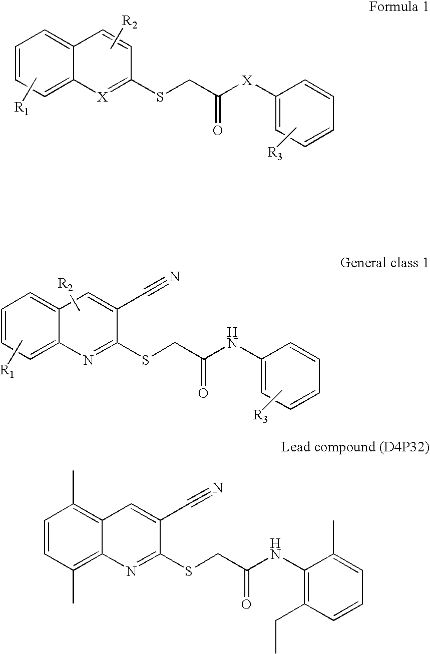

where each of R₁-R₃ is independently selected from the group consisting of a hydrogen atom, a halogen atom, a hydroxyl and a methoxyl group, and any other organic group containing any number of carbon atoms in a linear, branched, or cyclic structural format; and each of X is a heteroatom such as N, O, S, and C. More specifically the lead compound is D4P32.

In another embodiment, the invention relates to a compound, or a pharmaceutically acceptable salt, solvate, or hydrate thereof, wherein the compound comprises a general formula 2 from the general class 2,

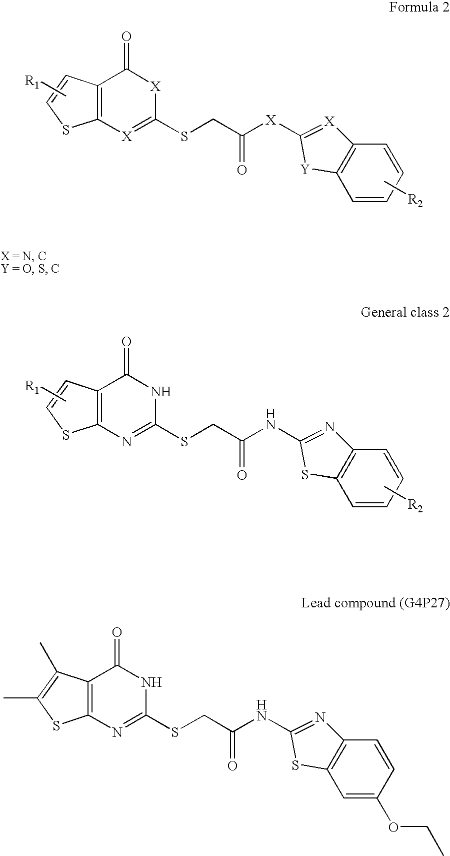

where each of R₁-R₂ is independently selected from the group consisting of a hydrogen atom, a halogen atom, a hydroxyl and a methoxyl group, and any other organic group containing any number of carbon atoms in a linear, branched, or cyclic structural format; and each of X or Y is a heteroatom such as N, O, S, and C. More specifically the lead compound is G4P27.

In a related embodiment, the invention relates to a compound, or a pharmaceutically acceptable salt, solvate, or hydrate thereof, wherein the compound comprises a general formula 3 from the general class 3,

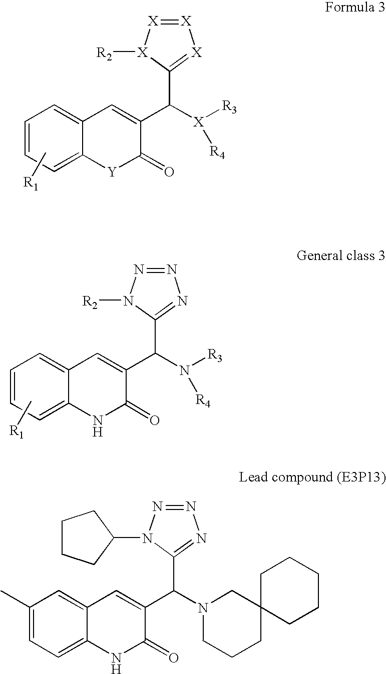

where each of R₁-R₄ is independently selected from the group consisting of a hydrogen atom, a halogen atom, a hydroxyl and a methoxyl group, and any other organic group containing any number of carbon atoms in a linear, branched, or cyclic structural format; and each of X or Y is a heteroatom such as N, O, S, and C. More specifically the lead compound is E3P13. Another embodiment of this claim is stereochemical mixtures or pure forms of E3P13 existing as either S or R configurations.

In yet another embodiment, the invention relates to A compound, or a pharmaceutically acceptable salt, solvate, or hydrate thereof, wherein the compound comprises a general formula 4 from the general class 4,

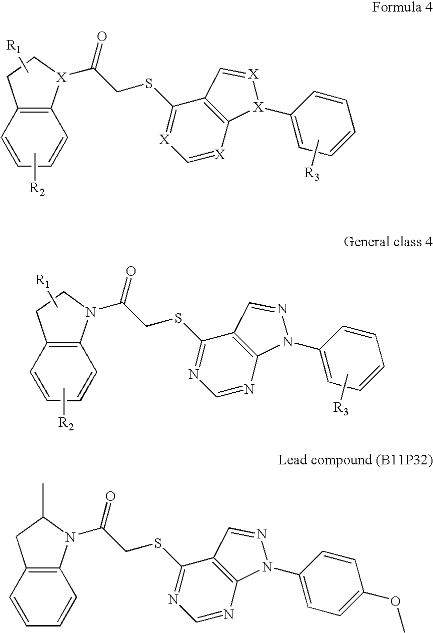

where each of R₁-R₃ is independently selected from the group consisting of a hydrogen atom, a halogen atom, a hydroxyl and a methoxyl group, and any other organic group containing any number of carbon atoms in a linear, branched, or cyclic structural format; and each of X is a heteroatom such as N, O, S, and C. More specifically the lead compound is B11P32.

In a closely related embodiment, the invention relates to A compound, or a pharmaceutically acceptable salt, solvate, or hydrate thereof, wherein the compound comprises a general formula 5 from the general class 5,

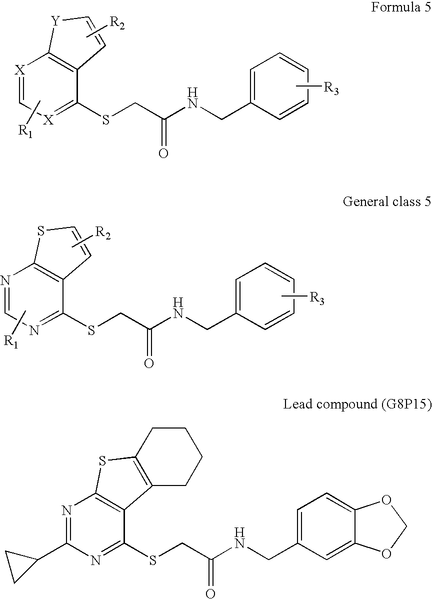

where each of R₁-R₃ is independently selected from the group consisting of a hydrogen atom, a halogen atom, a hydroxyl and a methoxyl group, and any other organic group containing any number of carbon atoms in a linear, branched, or cyclic structural format; and each of X or Y is a heteroatom such as N, O, S, and C. More specifically the lead compound is G8P15.

The above-mentioned and other features of this invention and the manner of obtaining and using them will become more apparent, and will be best understood, by reference to the following description, taken in conjunction with the accompanying drawings. The drawings depict only typical embodiments of the invention and do not therefore limit its scope.

### BRIEF DESCRIPTION OF THE FIGURES

FIG. 1. A high scoring docking poses of (R) and (S) configurations of E3P13 on the active site of LYP. Transparent surface area represents the substrate-binding pocket. Green stick model represents the compound E3P13. Side chain of major interacting amino acid residues are displayed in stick.

FIG. 2. Intracellular inhibition of PTPN22 by compound E3P13. Top panels, anti-pSrc (Tyr416) immunoblots of lysates of Jurkat TAg cells treated with 50 μM of the top five compounds in Table I (lanes 3 and 4 in each panel) or untreated (lanes 1 and 2 in each panel) and either left unstimulated (lanes 1 and 3 in each panel) or stimulated (lanes 2 and 4 in each panel) with C305 for 2 min. Bottom panels, anti-Lck blot of same samples. Treatment with E3P13 caused identical effects in three independent experiments. (B) Top panels, anti-pSrc (Tyr416) immunoblots of lysates of mouse thymocytes treated with 50 μM of compound E3P13 (lanes 3 and 4 in each panel) or untreated (lanes 1 and 2 in each panel) and either left unstimulated (lanes 1 and 3 in each panel) or stimulated (lanes 2 and 4 in each panel) with biotinylated anti-CD3 and anti-CD4, followed by cros-linking with streptavidin for 1.5 min. Bottom panels, anti-Lck blot of same samples. Figure is representative of two independent experiments. (C) Same experiment as in (B), but performed on thymocytes from PTPN22 KO mice. Arrows indicate the position of Lck in each panel.

FIG. 3. Treatment of T cells with E3P13 affects TCR signaling downstream Lck. (A) Jurkat TAg cells were treated with 50 μM of E3P13 (lanes 3 and 4 in each panel) or left untreated (lanes 1 and 2 in each panel) and were either left unstimulated (lanes 1 and 3 in each panel) or stimulated (lanes 2 and 4 in each panel) with C305 for 2 min. Panels show blots of total lysates with the following antibodies: top panel, anti-pSrc (Y416); second from top, control anti-Lck; third from top, anti-pZAP70 (Y319); third from bottom, control anti-ZAP70; second from bottom, antipERK; bottom panel, control anti-ERK. (B) Single-cell study TCR-induced phosphorylation of SLP-76 by phospho-flow cytometry. Jurkat TAg cells were treated with 50 μM of E3P13 (continuous line and black graphs) or left untreated (dashed-line and grey graphs) either left unstimulated (white graphs) or stimulated with C305 for 2 min (grey and black graphs). Cells were immediately fixed and stained with a PE-conjugated anti-pSLP-76 (Tyr128) antibody. Graphs show expression of pSLP-76 levels as detected by flow cytometry after gating on high forward and high side scatter (=cells with high SLP76 phosphorylation) cells. The statistical significance of the difference between E3P13-treated and untreated cells was calculated by Overton subtraction (32). Data are representative of two experiments with identical results.

### DETAILED DESCRIPTION OF THE INVENTION

The lymphoid tyrosine phosphatase LYP, encoded by the gene PTPN22, is a critical negative regulator of signaling through the T cell receptor. A gain-of-function R620W genetic polymorphism of LYP has recently emerged as an important risk factor for human autoimmunity. Development of a specific LYP inhibitor would be of wide spread use, both in elucidating the biological role(s) of LYP and as a potential therapeutic for human autoimmunity. Here, we screened a library consisting of a highly diverse set of 4000 compounds with an in vitro phosphatase assay, which resulted in the identification of a series of novel LYP inhibitors. In order to understand the underlying interaction between LYP and the compounds, we performed docking studies and analyzed the structure-activity relationship. Upon testing our inhibitors in cells we obtained a lead, which inhibited LYP and restored the early events of TCR signaling.

The following examples are intended to illustrate, but not to limit, the scope of the invention. While such examples are typical of those that might be used, other procedures known to those skilled in the art may alternatively be utilized. Indeed, those of ordinary skill in the art can readily envision and produce further embodiments, based on the teachings herein, without undue experimentation.

### Experimental Procedures

Materials, chemicals and enzymes. All chemicals were purchased from commercial sources unless otherwise noted. Compounds were dissolved in DMSO, and stock solutions were stored at −20° C. The catalytic domain of CD45 was purchased from Biomol Inc.

Antibodies and reagents. Anti-mouse-CD3 and anti-mouse-CD4 antibodies were purchased from BD Biosciences while the anti-human CD4 antibody was from Lab vision. Streptavidin was obtained from Sigma and the F(ab)₂ crosslinker was from Jackson Immunolabs. The polyclonal anti-pTyr319-Zap-70, anti ZAP-70, anti-pTyr505-Lck and anti-pTyr416-Src antibodies were obtained from Cell Signaling Technology, Inc., while the anti-Lck (3A5) and anti-ERK2 antibodies were from Santa Cruz Biotechnology. The Anti-ACTIVE MAPK polyclonal antibody was obtained from Promega. The PE-conjugated anti-pSLP76 antibody was purchased from BD (Carlsbad, Calif.). The ECL-Plus Chemiluminescence kit was obtained from GE-Amersham Biosciences.

Purification of recombinant proteins. The modified pBAD plasmid encoding the catalytic domain of HePTP (aa 44-339) in frame with a non-cleavable 6×His tag was a kind gift of Lutz Tautz (26). cDNA fragments encoding the catalytic domains of LYP (aa 2-309) and PTP-PEST (aa 2-323, PEST) were cloned between the BamH1 and the Xho1 sites of the pET28a plasmid (Novagen) in frame with a cleavable N-terminal 6×His-tag. Recombinant proteins were purified from lysates of IPTG-induced E. coli BL21 cells by affinity chromatography on Ni-nitrilotriacetic acid columns. 6×His HePTP was eluted using 250 mM imidazole. Untagged LYP and PTP-PEST were eluted by incubating columns with thrombin, followed by removal of thrombin from the protein preparation by a second chromatography step on benzamidine columns. LMPTP-A was cloned between the BamHI and the XbaI sites of the pEGST vector (27), which allows expression of recombinant proteins in fusion with an N-terminal GST and under control of the strong T7 promoter. The enzymes were expressed in BL21 E. coli cells and isolated from lysates of IPTG-induced bacteria by a single-step of affinity chromatography on glutathione-sepharose (GE Healthcare, Inc.) followed by elution with a glutathione solution.

Chemical Library Screening for LYP Inhibitors. A library consisting of a highly diverse set of 4,000 compounds was screened in a 96-well format in an assay specific for LYP activity. Each reaction contained 300 nM LYP, 2 mM p-NPP, and 40 μg/mL compound in a final volume of 60 μl of 50 mM BisTris pH 6.0 reaction buffer. The reaction mixture with the compounds was incubated for 25 min at 37° C. The reaction was stopped by addition of 504 of 1 M NaOH. The compounds were initially screened at 40 μg/mL and the reaction was initiated by addition of p-NPP and thereafter the enzyme. Compounds with an inhibition of 50% or more were further tested at multiple concentrations. All assays were done in triplicate. The IC₅₀ values were then determined for each drug from a plot of log [drug concentration] versus percentage of enzyme inhibition. Absorbance data were measured using a Molecular Devices Emax Precision Microplate reader.

Phosphatase assays. The activity of LYPCAT in the presence of E3P13 was detected using a phospho-tyrosine (pY) peptide ARLIEDNEpYTAREG, derived from the Lek Y394 phosphorylation site. All reactions were carried out at 37° C. in a buffer containing 50 mM Bis-Tris, pH 6.0, 1 mM DTT, amounting to a final volume of 50 μL. Each reaction contained 25 nM LYPCAT and 13.72 μM pY peptide (equal to the Km value). The amount of DMSO was held constant and did not exceed 6% of the total reaction volume. When the pY-peptide was used as substrate, the reaction was stopped by addition of BIOMOL GREEN™ (Biomol, Inc., Plymouth Meeting, Pa.) and the phosphate released was detected by measuring the absorbance at 595 nm. Time of reaction and amount of enzyme were optimized in order to ensure that the readings were taken in initial rate conditions. The phosphatase activity measured in triplicate was corrected for the non-specific signal of identical reactions performed also in triplicate without addition of enzyme. The 1050 values were then determined from a plot of inhibitor concentration versus percentage enzyme activity. Absorbance was measured on a Perkin Elmer 1420 Multilabel Counter Victor3 V plate reader (Perkin-Elmer, Turku, Finland). The data were plotted using GraphPad Prism®.

Lineweaver-Burk plots. The LYP-catalyzed hydrolysis of p-NPP was measured in the presence of various fixed concentrations of inhibitor at a series of substrate concentrations (ranging from 0.25 to 5 mM). Assays were conducted in triplicate at 37° C. in a reaction buffer consisting of 50 mM Bis-Tris, pH 6.0 and 1 mM DTT amounting to a final volume of 50 μL. The reaction was stopped by the addition of 100 μL 1 M NaOH. The activity was detected by monitoring absorbance of the product; para-nitrophenol at 405 nm and the amount of Product was determined using a molar extinction coefficient of 18,000 M-1 cm-1. The reciprocal of the reaction rate was plotted as a function of the reciprocal of the substrate concentration for each concentration of inhibitor. The data were plotted using GraphPad Prism®.

Docking Studies. The docking studies were carried out by the GOLD (version 3.2 Genetic Optimization for Ligand Docking) software package (28) running on our multi-processor linux PC and a 24-processor Silicon Graphics Onyx workstation as described. We used the four crystal structure complexes bound with a ligand as the starting structure, where the ligand was subsequently removed to keep the binding pocket available (29). Likewise, all the water molecules present in the crystal structure were removed. Hydrogen atoms were added to both protein and ligand according to the calculated protonation state at pH 7.0 during the docking studies. The selected X-ray complexes included low-molecular PTP LMPTP (PDB: 1PHR), LYP (PDB: 2QCJ), D1 domain of the receptor protein PTP CD45 bound with tyrosin based activation peptide (PDB: 1YGR), and an ancestral 3-deoryald-2-ulosonate-phosphate synthase (PDB: 1ZCO). In each structure a spherical area surrounding the ligand-binding site with a radius of 10 Å was selected for docking. The ligand was then placed within the active site using the least-square fitting procedure applied in GOLD program. All docking runs were performed using standard default settings with a population size of 100, a maximum number of 100,000 operations, and a mutation and crossover rate of 95. The scoring function was contributed from H-bond interactions, van der Waals interactions within the complex, and the ligand internal energy summarized by ligand steric and torsional energies (28).

Cells and Cell treatments. Jurkat T leukemia cells expressing SV-40 large T Antigen (JTAg) (30) were kept at logarithmic growth in RPMI 1640 medium supplemented with 10% fetal bovine serum, 2 mM L-glutamine, 1 mM sodium pyruvate, 10 mM HEPES pH 7.3, 2.5 mg/mL D-glucose, and 100 units/mL of penicillin and 100 μg/mL streptomycin. Fixed concentrations of inhibitors (50 μM) or DMSO (control) were added to 20×10⁶ cells suspended in 800 RPMI 1640, and incubated for 1 h at room temperature. The volume of DMSO added was held constant at less than 2% of the total volume. JTAg cells pre-incubated with DMSO or inhibitor were divided into 400 μL aliquots containing 10×10⁶ cells and stimulated with supernatants of C305 hybridoma (31) for 2 min or left untreated. Cells were lysed in 20 mM Tris-HCl pH 7.5, 150 mM NaCl, 5 mM EDTA pH 8.0 containing 1% NP-40, 10 μg/mL aprotinin and leupeptin, 10 μg/mL soybean trypsin inhibitor, 1 mM Na3VO4 and 1 mM phenylmethylsulfonyl fluoride after which lysates were clarified by centrifugation at 13,200 rpm for 20 minutes. The total protein concentration in each cell lysate was determined by the Bradford protein assay (Bio-Rad) in order to normalize the amount of protein used in SDS-PAGE.

Thymocytes were isolated from the homogenized thymi of 4-6 week old C57BL/6 (PTPN22+/+) mice (Taconic Farms, Inc.) or PTPN22−/− (Genentech, Inc.) after depletion of red blood cells with lysis buffer following standard procedures. A fixed concentration of E3P13 (50 μM) or DMSO (control) was added to 20×10⁶ cells suspended in 800 μL RPMI 1640, and incubated for 1 h at room temperature. The volume of DMSO added was held constant at less than 2% of the total volume. Thymocytes pre-incubated with DMSO or inhibitor were divided into 200 μL aliquots containing 10×10⁶ cells, treated with biotinylated CD3 and CD4 antibodies for 30 min and stimulated with the crosslinker, streptavidin for 1.5 min, or left untreated. Cells were lysed in 20 mM Tris-HCl pH 7.5, 150 mM NaCl, 5 mM EDTA pH 8.0 containing 1% NP-40, 10 μg/mL apoprotein and leupeptin, 10 μg/mL soybean trypsin inhibitor, 10 mM Na₃VO4 and 1 mM phenylmethylsulfonyl fluoride after which lysates were clarified by centrifugation at 13,200 rpm for 20 minutes.

Aliquots of lysates were suspended in SDS sample buffer, heated at 95° C. for 5 minutes and the boiled samples run on 10% SDS-polyacrylamide gels. Proteins resolved by gel electrophoresis were transferred onto nitrocellulose membranes (Hybond ECL, GE Healthcare), using appropriate dilutions of unconjugated primary antibodies followed by HRP-conjugated secondary antibodies (purchased from GE Healthcare). Blots were developed with the enhanced chemiluminescence detection system, ECL-Plus, following manufacturers directions.

Phosphoflow cytometry. Phospho-flow cytometry was performed on JTAg cells following published protocols (BD Biosciences). Cells were treated with 50 μM E3P13 or left untreated, fixed with BD Cytofix buffer and permeabilized using BD Phosflow III and stained with PE-conjugated anti-pSLP76 (Tyr128) antibody. Cell fluorescence was assessed on a Cytomics FC500 cytometer (Beckman Coulter) at the USC Immune Monitoring Core. Data Analysis and graph preparation were carried using FlowJo (Tree Star, Inc., Ashland, Oreg.). Differences between distributions were calculated by Overton subtraction (32) The sampling error in 0305-stimulated JTAg cells was evaluated by measuring the average±SD Overton subtraction % of three independent replicates and was 2.5±0.6% for the anti-pSLP-76 antibody.

### Results

Identification of Compounds as LYP Inhibitors. We initially screened a library of 4000 drug-like small-molecule compounds against LYP/PTPN22, using p-nitrophenyl phosphate (pNPP) as a substrate. The ability of the library members to inhibit LYP/PTPN22 catalyzed hydrolysis of pNPP was measured in a buffer containing 50 mM Bis-Tris pH 6.0, 1 mM DTT at 37° C. A total of 137 compounds exhibited >50% inhibition at a concentration of 40 μg/mL and were further tested at different concentrations to calculate IC₅₀ values. The LYP inhibitory activity of these 137 compounds ranged from 11-100 μM. We selected the top five inhibitors that exhibited 1050 values of <25 μM for closer inspection. Table I lists the structures of these compounds.

The top compounds were counter-screened with a panel of other phosphatases including PTP-PEST, HePTP, LMPTP-A and CD45. As seen from their inhibition data in Table II, the 1050 values for the best compounds range from 11-30 μM. In general, these compounds were equally potent against LYP and HePTP, inactive against LMPTP-A and CD45, and moderately active against PTP-PEST. Further structural optimizations are underway in our laboratory to increase potency and selectivity for LYP.

The effect of E3P13 on LYP was then assessed using a 14-amino acid peptide, ARLIEDNEpYTAREG, derived from the Y394 phosphorylation site of Lek, a physiological substrate of LYP. This peptidic substrate interacts with a wider PTP surface and not just the catalytic pocket, thus translating to a higher specificity for the enzyme. It therefore follows that the 1050 value obtained from this assay would be more accurate reflection of the concentration of the inhibitor to be used in cell-based assays. An IC₅₀ of 60 μM was obtained using the pY peptide and concentrations close to this value were used to test the effect of E3P13 in cells. The Lineweaver-Burk plot of E3P13 suggests a mixed type of inhibition (data not shown).

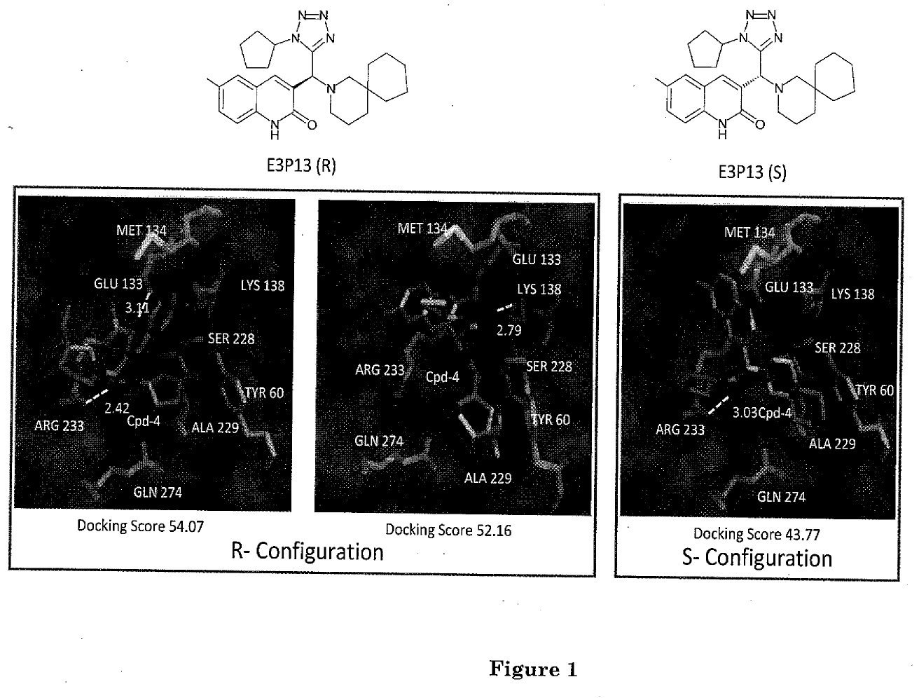

Docking Studies. To better understand the nature of selectivity of these inhibitors we docked the top five compounds on the ligand-binding site of LMPTP-A (PDB: 1PHR), LYP (PDB: 2QCJ), D1 domain of the receptor protein PTP CD45 bound with tyrosine based activation peptide (PDB: 1YGR), and an ancestral 3-deoryald-2-ulosonate-phosphate synthase (PDB: 1ZCO). Table III summarizes the predicted scores of the top five compounds from docking simulations against each target. Higher GOLD-generated scores are indicative of better fits and in general our docking scores were in agreement with the enzymatic activities against selected phoshphatase. Compound D4P32 and G4P27 showed the best docking scores against LYP and poorest scores against LMPTP-A. Compound E3P13 with a methine chiral center is a mixture of two stereoisomers. Docking studies also predicted significantly different scores with each isomer, suggesting that both the stereochemistry and ring conformation are critical in mapping the ligand-binding mode. Both R and S isomers scored the best against LYP, worst against LMPTP-A and with intermediate scores against the other two phophatases, CD45 and HePTP. FIG. 1 shows the possible binding mode of (S)-E3P13 against LYP. Both K136 and K138 form H-bonds with the tetrazole motif, while most of the nearby residues form van der Waals interactions with the ligand. A part of the quinolinone motif is solvent exposed but the orientation might be influenced by the π-π interactions with Y60.

Compound B11P32 is also a mixture of two isomers. Although this compound showed identical IC₅₀ values against LYP and HePTP, there was a significant difference in the selectivity of R versus S isomers in our docking studies. For example, the R isomer gave a score of 67.3 and S isomer a score of 57.9 against LYP. On the contrary, the S isomer (score of 72.9) gave a better score than the R isomer (score of 64.1) against HePTP. To better address the selectivity issues with different stereoisomers, we are in the process of synthesizing both isomers of B11P32 and E3P13. These studies will be reported in due course.

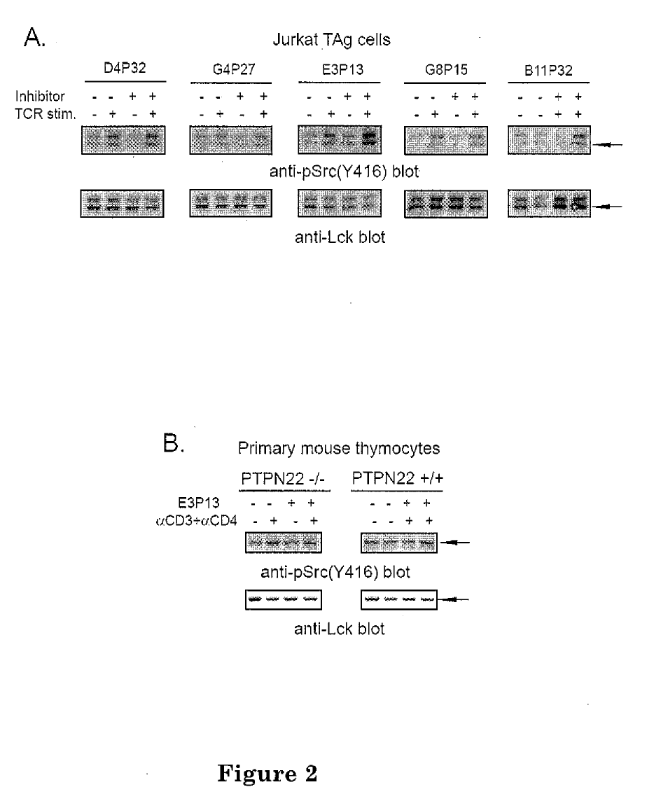

Effect of inhibitors on TCR Signaling in cells. Next, we tested the top five inhibitors for their ability to inhibit LYP in cells. The five compounds D4P32, G4P27, E3P13, B11P32 and G8P15 were chosen on the basis of their potency as well as their selectivity for LYP over the other phosphatases investigated (Table II). First, we tested the effects of the five compounds on the early stages of TCR signaling in the Jurkat T Antigen (JTAg) human T cell line. As a read-out of PTP activity, we analyzed the phosphorylation of Lck, a protein tyrosine kinase (PTK) involved in the initial stages of TCR signaling, at its positive regulatory Tyr394 residue. Lck is a well-known physiological substrate of the PTPs CD45 and LYP, through its two major phosphorylation sites at Tyr505 and Tyr394, respectively (33,34). Tyr394 of Lck is also dephosphorylated by PTP-PEST in T cells (35). Following incubation with LYP inhibitors (50 μM of D4P32, G4P27, E3P13, G8P15 and B11P32) for 1 h, we stimulated JTAg cells with C305 antibodies for 2 min at 37° C. Lysates of these cells were probed with anti-pSrc (Tyr416), an antibody which selectively recognizes the phosphorylated Tyr416 in Src, and in T cell lysates cross-reacts with the equivalent pTyr394 site of Lck. Anti-pSrc (Tyr416) immunoblots of the lysates showed that among the five inhibitors tested, E3P13 increased phosphorylation at 56 kDa, (FIG. 2, top panel) relative to the respective controls corresponding to higher phosphorylation of Lck at Tyr394. However, D4P32, G4P27, G8P15 and B11P32 do not increase Lck phosphorylation at this site (FIG. 2A, top panel; anti-Lck control blots, bottom panel). Thus, the five inhibitors tested have different efficacies in JTAg cells, and among them, E3P13 affects TCR signaling in a manner that is concordant with the inhibition of LYP/PTPN22.

To extend this observation to primary T cells and to directly test if the increase in Tyr394 phosphorylation was due to PTPN22 inhibition, we analyzed the effect of E3P13 on thymocytes of PTPN22−/− knockout mice. In parallel, the compound was also tested on T cells from normal PTPN22+/+ mice. Freshly isolated thymocytes from PTPN22−/− and PTPN22+/+ mice were incubated with 50 μM of E3P13 for 1 h, stimulated with anti-CD3 and anti-CD4 antibodies, plus a crosslinker (streptavidin) for 1 min at 37° C. As seen in FIG. 2B, anti-pSrc (Tyr416) immunoblots of thymocyte lysates from PTPN22−/− mice, indicate that E3P13 had no effect on phosphorylation of Lck at Tyr394. This suggests that E3P13 preferentially inhibits PTPN22 over other phosphatases. In agreement with this notion, when thymocytes isolated from normal PTPN22+/+ mice were incubated with E3P13, anti-pSrc416 immunoblots of the lysates of these cells demonstrated increased Lck phosphorylation at Tyr394 (FIG. 2B).

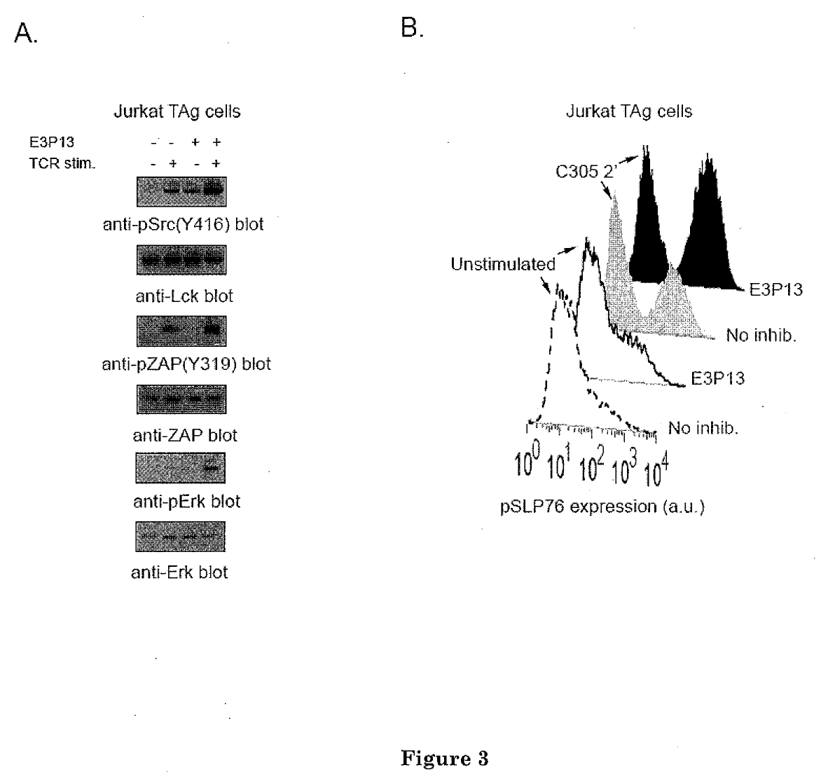

Effect of E3P13 on events downstream of Lck-Given the observed increase of Lck Tyr394 phosphorylation (FIG. 3A, top panels) due to E3P13, we proceeded to evaluate its effect on downstream signaling events such as the activation of ZAP-70 and on the Ras-Raf-MAPK pathway (ERK112) in JTAg cells. JTAg cells were incubated with E3P13 (50 μM) for 1 h and subsequently stimulated and lysed. In agreement with the results observed with Lck, E3P13 increased TCR-stimulated phosphorylation of ZAP-70, as seen from the anti-pZAP (Tyr319) blot (FIG. 3A, middle panels). In addition, E3P13 also increased ERK1/2 phosphorylation, as evidenced by the anti-pERK1/2 blot (FIG. 3A, lower panels). These results suggest that E3P13 restores TCR signaling by inhibiting LYP/PTPN22.

To provide further evidence for our notion, we assessed the effect of the inhibitor on TCR-induced phosphorylation of SLP-76, at the single cell level by phospho-specific flow cytometry (FIG. 3B). SLP-76 is an early mediator of TCR signaling downstream of Lck and LYP. JTAg cells were incubated with 0 μM (control) or 50 μM E3P13 for 1 h, subsequently stimulated or left untreated and stained with PE-conjugated anti-pSLP76 antibody. Compared to the control (gray graph), as seen in FIG. 3B, cells treated with E3P13 showed higher levels of TCR-induced SLP-76 phosphorylation (black graph). Taken together, all these experiments demonstrate that E3P13 is efficient in restoring TCR signaling by suppressing the function of LYP/PTPN22.

### Discussion

LYP/PTPN22 is an important target in the treatment of human autoimmunity. However, only a few potent/selective LYP inhibitors have been described (36-38). On account of the highly conserved active site, many PTP inhibitors are phosphotyrosine mimics (36,37,39). The goal of the present study was to develop specific cell-permeable, small-molecule LYP inhibitors using a combination of chemical library screening, virtual docking and cell-based assays. This approach resulted in the identification of compounds that inhibit LYP with efficacies ranging from 11-30 μM. The identification of these lead structures will aid in the rational design of future inhibitors.

The inhibitors reported herein differ structurally from the majority of other LYP inhibitors in that they are not salicylic acid derivatives, a moiety often used as a phosphotyrosine analog (36). A closer inspection of their structures reveals that the compounds are neutral in charge compared to these and are therefore expected to be more cell-permeable. Interestingly, upon testing our top 5 inhibitors in JTAg cells, our results demonstrated that amongst them E3P13 alone significantly increased Lck Tyr394, ZAP-70, SLP-76 and ERK1/2 phosphorylation. A plausible reason for the different inhibitory activities of the compounds towards LYP in JTAg cells could be due to the inhibition of CD45. Another possible rationale for the varying efficacies could be the differential uptake of the compounds by the cells. Key evidence that E3P13 selectively inhibits PTPN22 was obtained from experiments with cells from PTPN22−/− mice. The findings from this clearly indicate that Lck phosphorylation at Tyr394 is not enhanced in thymocytes from the knockout mice. However, it must be pointed out that while E3P13 is selective, rational structural modifications are needed to increase its potency in order to conduct injection studies in the future.

The docking studies showed that all the top five compounds scored high against LYP and very low against LMPTP-A with intermediate scores against the other two phosphatases. There was a significant difference between the R and S stereoisomers when docked against LYP and HePTP, even though in vitro data indicate identical enzyme inhibition. These studies imply that selective and potent LYP inhibitors can be designed based on these novel scaffolds. Our model also provides a reasonable platform for an extensive structure-based drug design targeting phosphatases.

In summary, we have identified a series of LYP inhibitors with low molecular weight and non-peptidic features showing the potency of being good leads through chemical screening using an assay specific for LYP activity. Cell studies of the promising inhibitors led to the validation of a hit that restored TCR signaling in the JTAg cell line. We have confirmed lead compound selectivity in primary mouse thymocytes. Finally, from the lead compounds we have identified 80 analogues that may have promising therapeutic potential. Activity, selectivity, and docking scores of the 80 analogues of E3P13 are presented in Table 2. All compounds were also tested against a homologous protein tyrosine phosphatase called PEST.

The entire disclosure of each reference cited in this disclosure are relied upon and incorporated by reference herein.

TABLE I

| No. | ID # | Structure |
|---|---|---|
| 1. | D4P32 | 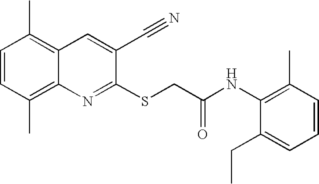 |
| 2. | G4P27 | 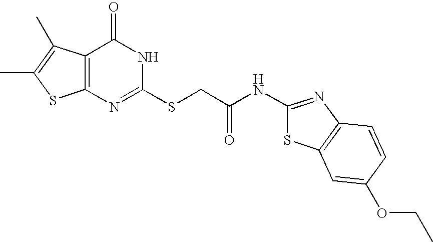 |
| 3. | E3 P13 | 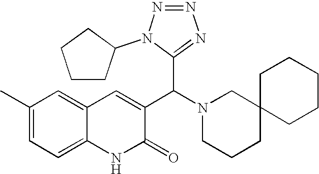 |
| 4. | B11P32 | 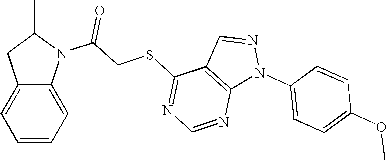 |
| 5. | G8P15 | 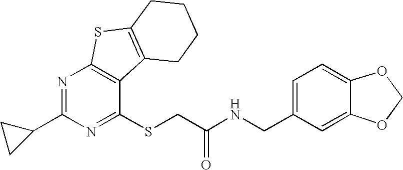 |

TABLE II

| | | PTP- | | | |
|---|---|---|---|---|---|
| | LYP | PEST | HePTP | LMPTP-A | CD45 |
| Compound | IC₅₀ (μM) | IC₅₀ (μM) | IC₅₀ (μM) | IC₅₀ (μM) | IC₅₀ (μM) |
| D4P32 | 11 | 35 ± 5 | 10 ± 2 | 72 | 11 |
| G4P27 | 11 ± 1 | 20 ± 3 | 9 ± 1 | >90 | 90 |
| E3 P13 | 20 ± 2 | >40 | 20 ± 2 | >90 | 87 ± 1 |
| B11P32 | 22 | >40 | 18 ± 3 | >90 | >90 |
| G8P15 | 22 ± 2 | 39 ± 1 | >40 | >90 | >90 |

TABLE III

| | LYP | CD45 | HePTP | LMPTP A |
|---|---|---|---|---|
| Compound | (2QCJ) | (1YGR) | (1ZCO) | (1PHR) |
| D4P32 | 59.1 | 54.45 | 56.3 | 41.8 |
| G4P27 | 64.0 | 57.30 | 60.3 | 51.7 |
| | R1: 47.9 | R1: 38.85 | R1: −8.4 | R1: −74.8 |
| E3P13* | S1: 51.8 | S1: 48.51 | S1: 17.5 | S1: −12.3 |
| | R2: 54.5 | R2: 40.82 | R2: 25.4 | R2: −27.8 |
| | S2: 63.0 | S2: 31.44 | S2: 12.5 | S2: −19.6 |
| B11P32* | R: 67.3 | R: 59.45 | R: 64.1 | R: −19.6 |
| | S: 57.9 | S: 52.95 | S: 72.9 | S: −21.8 |
| G8P15 | 55.5 | 55.14 | 74.7 | 34.4 |

*R and S represent stereochemistry, R1, R2 indicate the two ring conformations in the R isomer, and S1, S2 represent the two ring conformations in the S isomer.

TABLE IV

| Mol_ID | Structure | LYP IC50 | PEST IC50 | PEST_LYP | Docking score |
|---|---|---|---|---|---|
| 1 | 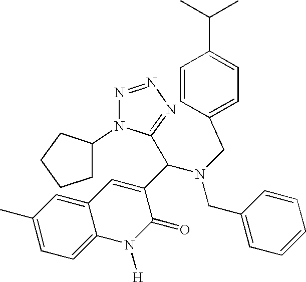 | 5.29 +/− 0.11 | 5.55 +/− 0.08 | 1.05 | 29.28 |
| 2 | 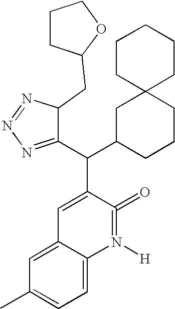 | 6.07 +/− 0.32 | 9.78 +/− 0.12 | 1.61 | 30.32 |
| 3 | 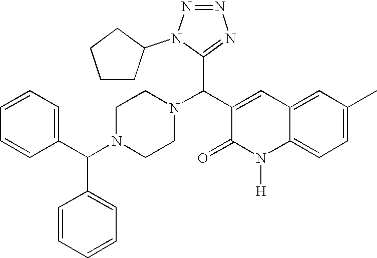 | 6.30 +/− 0.05 | 8.71 +/− 0.18 | 1.38 | 42.07 |
| 4 | 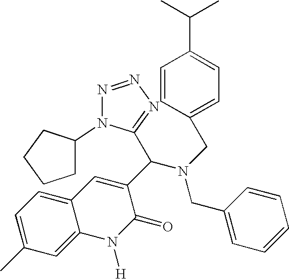 | 7.62 +/− 0.11 | 9.96 +/− 0.08 | 1.31 | 29.97 |
| 5 | 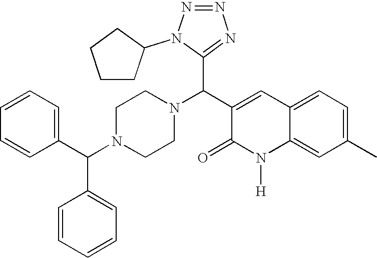 | 7.86 +/− 0.14 | 9.66 +/− 0.10 | 0.81 | 27.26 |
| 6 | 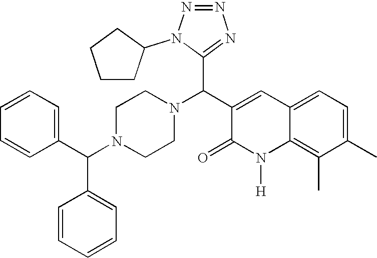 | 9.13 +/− 0.19 | 9.80 +/− 0.27 | 1.07 | 30.2 |
| 7 | 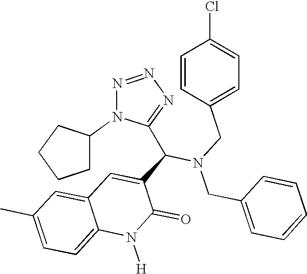 | 9.19 +/− 0.07 | 17.00 +/− 0.04 | 1.85 | 38.83 |
| 8 | 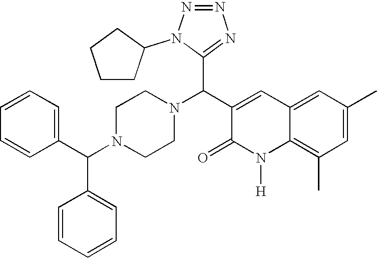 | 9.72 +/− 0.04 | 9.57 +/− 0.11 | 0.98 | 23.67 |
| 9 | 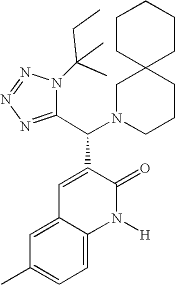 | 10.41 +/− 2.46 | 15.93 +/− 0.16 | 1.53 | 20.11 |
| 10 | 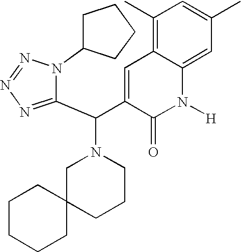 | 11.11 +/− 0.06 | 16.03 +/− 0.05 | 1.44 | 28.12 |
| 11 | 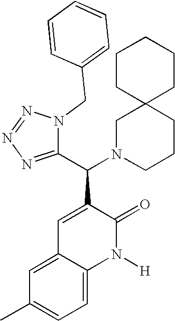 | 11.14 +/− 0.14 | 15.13 +/− 0.06 | 1.36 | 42.52 |
| 12 | 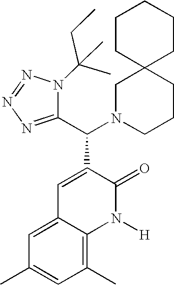 | 11.91 +/− 0.08 | 20.12 +/− 0.04 | 1.69 | 14.54 |
| 13 | 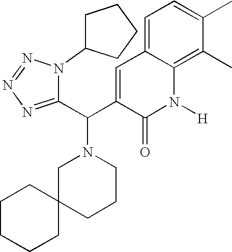 | 12.12 +/− 0.04 | 16.13 +/− 0.11 | 1.33 | 19.18 |
| 14 | 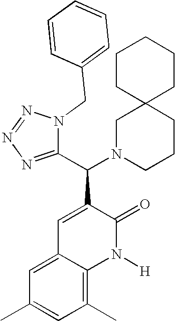 | 12.90 +/− 0.09 | 15.22 +/− 0.12 | 1.18 | 41.34 |
| 15 | 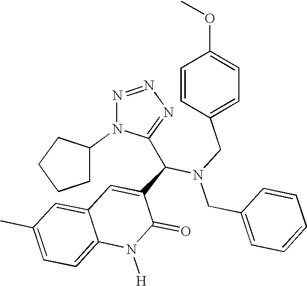 | 13.03 +/− 0.04 | 15.13 +/− 0.06 | 1.16 | 43.94 |
| 16 | 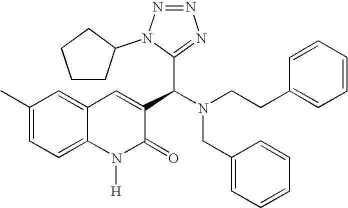 | 13.30 +/− 0.03 | 19.69 +/− 0.13 | 1.46 | 37.51 |
| 17 | 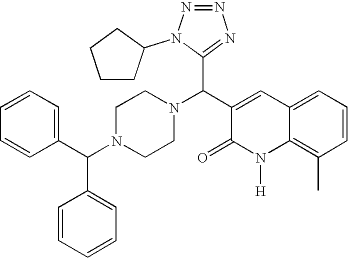 | 13.33 +/− 0.03 | 10.11 +/− 0.10 | 0.76 | 30.49 |
| 18 | 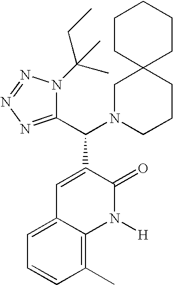 | 13.37 +/− 0.14 | 12.86 +/− 0.22 | 0.96 | 13.53 |
| 19 | 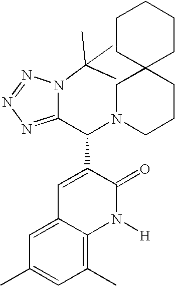 | 13.60 +/− 0.22 | 30.83 +/− 0.06 | 2.27 | 21.54 |
| 20 | 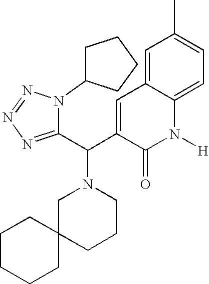 | 13.85 +/− 0.10 | 17.87 +/− 0.12 | 1.29 | 43.77 |
| 21 | 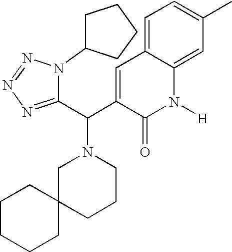 | 15.17 +/− 0.04 | 21.76 +/− 0.13 | 1.43 | 28.57 |
| 22 | 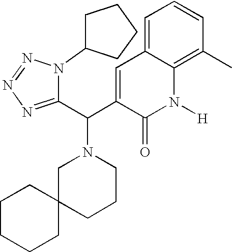 | 15.75 +/− 0.08 | 21.14 +/− 0.11 | 1.34 | 27.8 |
| 23 | 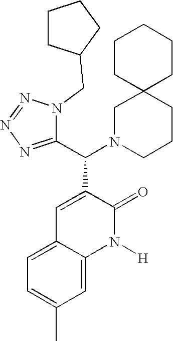 | 17.12 +/− 0.05 | 37.99 +/− 0.06 | 2.22 | 27.23 |
| 24 | 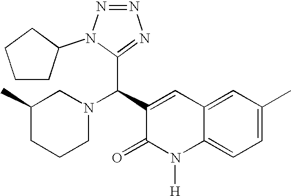 | 17.12 +/− 0.14 | 26.55 +/− 0.51 | 1.56 | 33.97 |
| 25 | 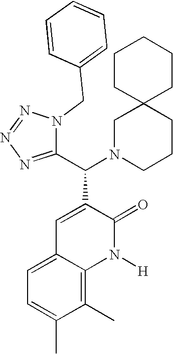 | 18.08 +/− 0.04 | 17.82 +/− 0.12 | 0.99 | 38.12 |
| 26 | 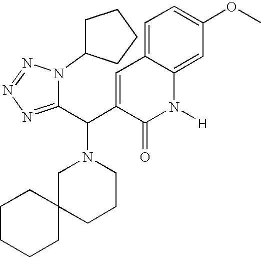 | 18.16 +/− 0.02 | 31.66 +/− 0.06 | 1.74 | 28.98 |
| 27 | 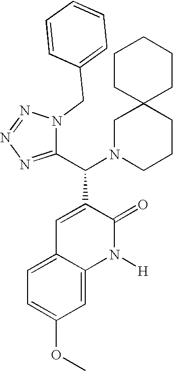 | 19.32 +/− 0.09 | 13.66 +/− 0.16 | 0.71 | 43.87 |
| 28 | 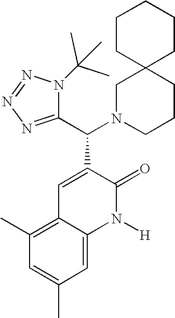 | 19.65 +/− 0.26 | 26.13 +/− 0.10 | 1.33 | 18.76 |
| 29 | 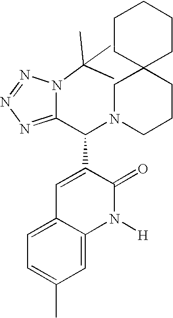 | 20.34 +/− 0.18 | 27.13 +/− 0.14 | 1.33 | 18.97 |
| 30 | 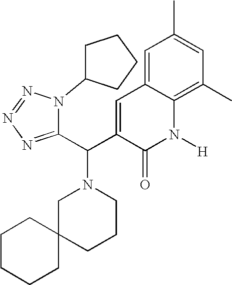 | 20.35 +/− 0.12 | 31.06 +/− .062 | 1.53 | 27.88 |
| 31 | 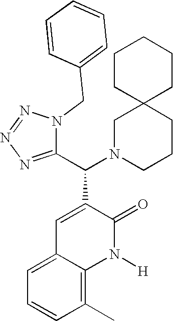 | 20.44 +/− 0.14 | 29.83 +/− 0.07 | 1.46 | 41.61 |
| 32 | 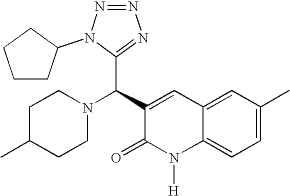 | 20.58 +/− 0.11 | 31.28 +/− 0.08 | 1.52 | 33.83 |
| 33 | 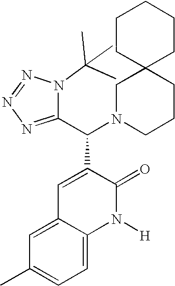 | 21.24 +/− 0.15 | 28.16 +/− 0.15 | 1.33 | 17.23 |
| 34 | 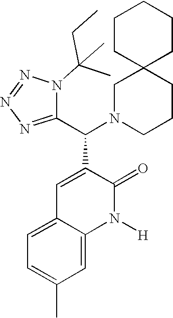 | 21.40 +/− 0.16 | 34.18 +/− 0.08 | 1.6 | 21.95 |
| 35 | 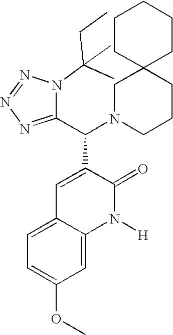 | 22.53 +/− 1.67 | 29.93 +/− 0.20 | 1.33 | 17.59 |
| 36 | 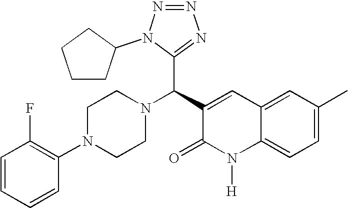 | 25.09 +/− 0.09 | 23.16 +/− 0.24 | 0.92 | 30.07 |
| 37 | 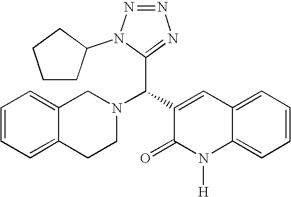 | 27.99 +/− 0.14 | 31.55 +/− 0.18 | 1.13 | 26.23 |
| 38 | 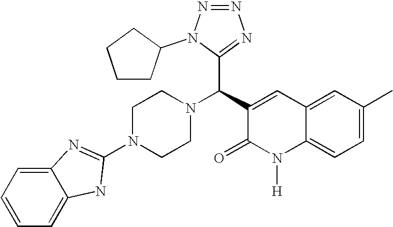 | 30.06 +/− 0.08 | 38.58 +/− 0.08 | 1.26 | 37.58 |
| 39 | 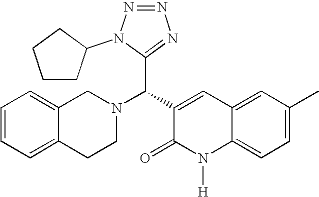 | 30.54 +/− 0.12 | 39.00 +/− 0.22 | 1.28 | 35.46 |
| 40 | 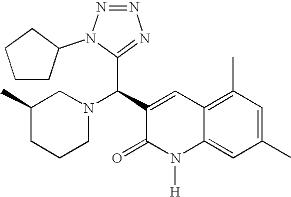 | 30.62 +/− 0.11 | 34.43 +/− 0.22 | 1.12 | 29.19 |
| 41 | 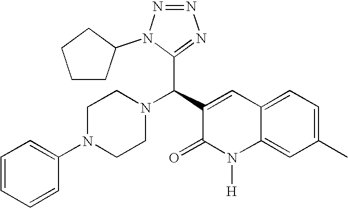 | 31.02 +/− 1.78 | ~36.5 | ~1.18 | 24.58 |
| 42 | 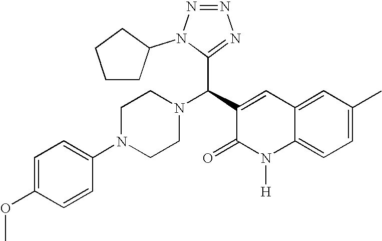 | 31.74 +/− 0.11 | 40.45 +/− 0.17 | 1.27 | 30.26 |
| 43 | 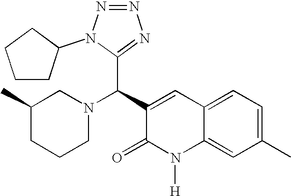 | 31.90 +/− 5.17 | 80.26 +/− 0.08 | 2.52 | 34.46 |
| 44 | 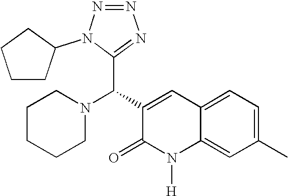 | 32.50 +/− 0.07 | 68.54 +/− 0.05 | 2.11 | 29.06 |
| 45 | 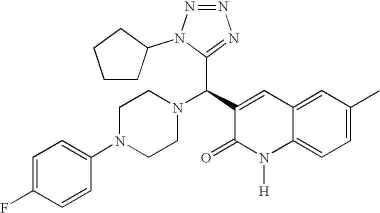 | 32.79 +/− 1.51 | 38.00 +/− 0.20 | 1.16 | 35.18 |
| 48 | 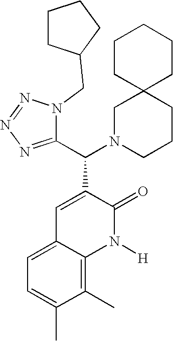 | 32.87 +/− 0.34 | 31.28 +/− 0.10 | 0.95 | 29.89 |
| 47 | 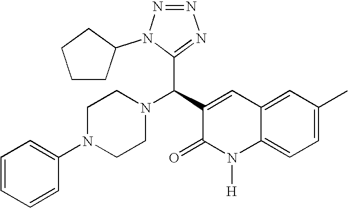 | 36.47 +/− 0.07 | 32.46 +/− 0.20 | 0.89 | 24.61 |
| 48 | 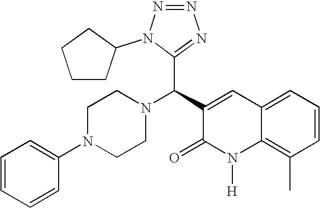 | 36.55 +/− 0.51 | 42.08 +/− 0.08 | 1.15 | 20.4 |
| 49 | 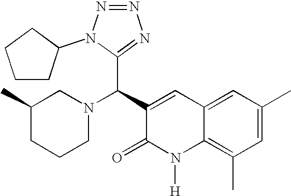 | 39.52 +/− 1.12 | 45.5 +/− 0.10 | 1.15 | 32.3 |
| 50 | 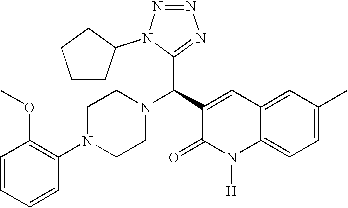 | >40 | . | . | 37.53 |
| 51 | 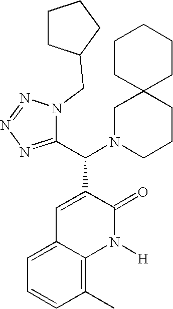 | >40 | . | . | 31.14 |
| 52 | 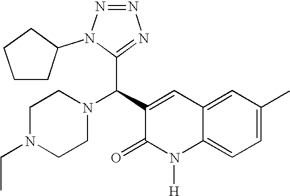 | >40 | . | . | 30.72 |
| 53 | 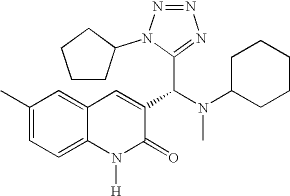 | >40 | . | . | 19.33 |
| 54 | 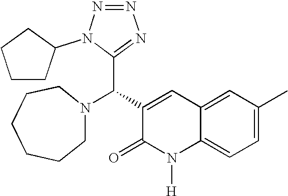 | >40 | . | . | 30.59 |
| 55 | 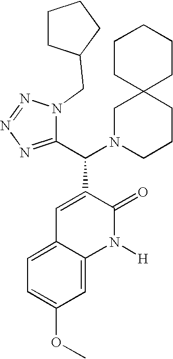 | >40 | . | . | 31.72 |
| 56 | 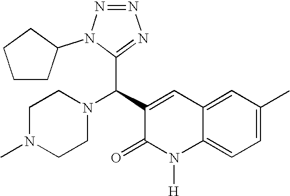 | >40 | . | . | 31.79 |
| 57 | 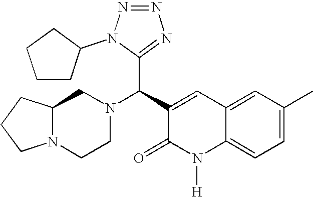 | >40 | . | . | 38.76 |
| 58 | 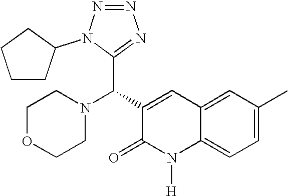 | >40 | . | . | 28.57 |
| 59 | 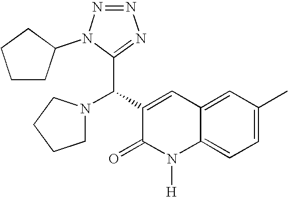 | >40 | . | . | 28.81 |
| 60 | 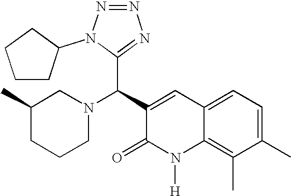 | >40 | . | . | 27.9 |
| 61 | 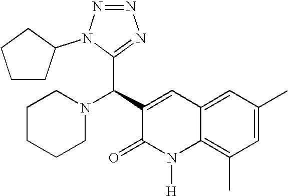 | >40 | . | . | 30.91 |
| 62 | 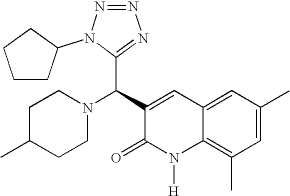 | >40 | . | . | 31.12 |
| 63 | 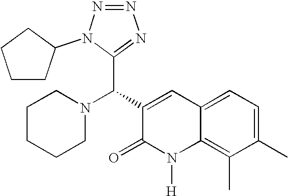 | >40 | . | . | 23.17 |
| 64 | 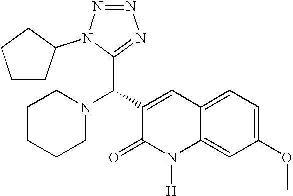 | >40 | . | . | 28.08 |
| 65 | 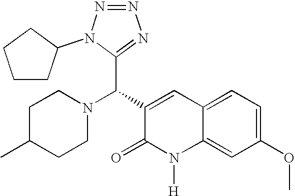 | >40 | . | . | 29.55 |
| 66 | 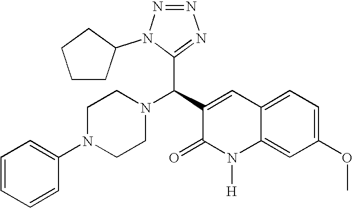 | >40 | . | . | 28.83 |
| 67 | 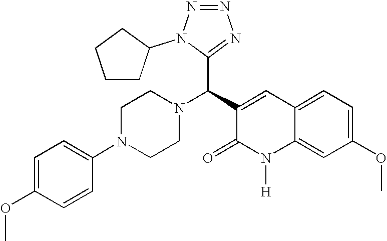 | >40 | . | . | 28.33 |
| 68 | 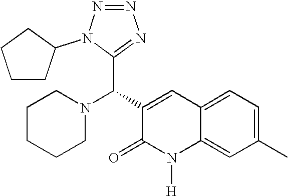 | >40 | . | . | 23.18 |
| 69 | 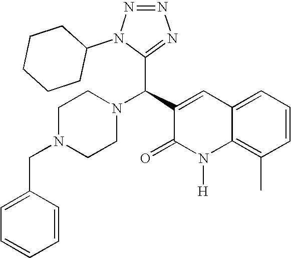 | >40 | . | . | 40.46 |
| 70 | 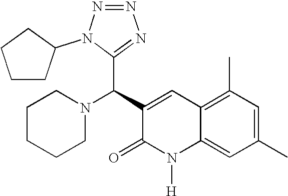 | >40 | . | . | 23.99 |
| 71 | 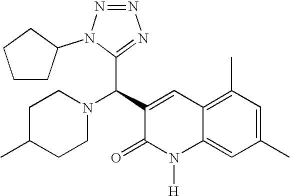 | >40 | . | . | 29.66 |
| 72 | 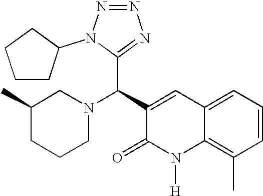 | >40 | . | . | 32.46 |
| 73 | 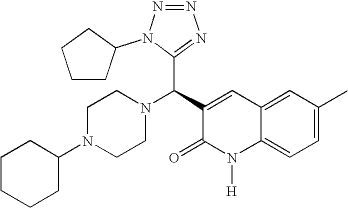 | >40 | . | . | 15.91 |
| 74 | 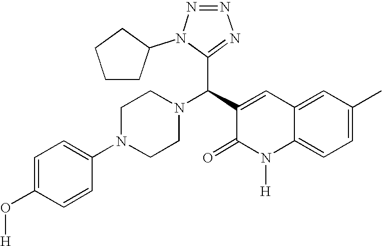 | >40 | . | . | 30.58 |
| 75 | 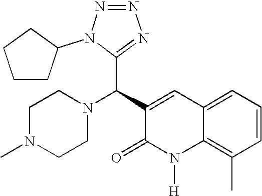 | >40 | . | . | 31.38 |
| 76 | 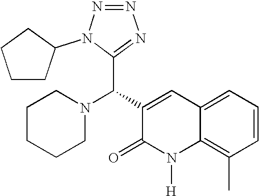 | >40 | . | . | 28.73 |
| 77 | 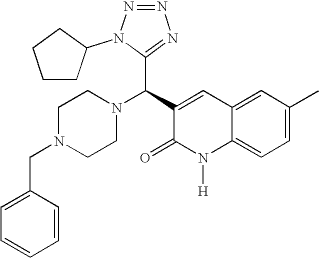 | >40 | . | . | 38.84 |
| 78 | 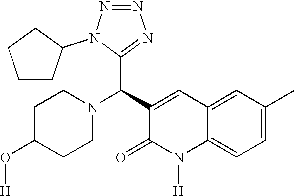 | >40 | . | . | 34.06 |
| 79 | 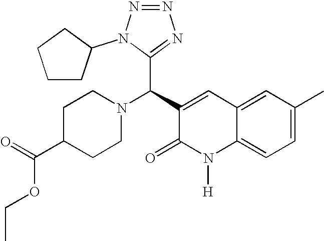 | >40 | . | . | 35.42 |
| 80 | 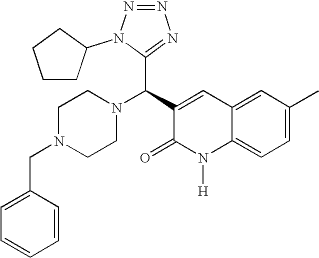 | >40 | . | . | 34.33 |

### REFERENCES

1. Christen, U., and von Herrath, M. G. (2004) Curr. Opin. Immunol. 16, 759-767
2. Goodnow, C. C., Sprent, J., Fazekas de St Groth, B., and Vinuesa, C. G. (2005) Nature 435, 590-597
3. Criswell, L. A., Pfeiffer, K. A., Lum, R. F., Gonzales, B., Novitzke, J., Kern, M., Moser, K. L., Begovich, A. B., Carlton, V. E., Li, W., Lee, A. T., Ortmann, W., Behrens, T. W., and Gregersen, P. K. (2005) Am J Hum Genet 76, 561-571
4. Carter, J. D., Calabrese, G. M., Naganuma, M., and Lorenz, U. (2005) J. Immunol. 174, 6627-6638
5. Kane, L. P., Lin, J., and Weiss, A. (2000) Curr. Opin. Immunol. 12, 242-249
6. Mustelin, T., Abraham, R. T., Rudd, C. E., Alonso, A., and Menlo, J. J. (2002) Front. Biosci. 7, d918-969
7. Siminovitch, K. A. (2004) Nat. Genet. 36, 1248-1249
8. Hermiston, M. L., Xu, Z., and Weiss, A. (2003) Annu Rev Immunol 21, 107-137
9. Vogel, A., Strassburg, C. P., and Manns, M. P. (2003) Genes Immun. 4, 79-81
10. Bottini, N., Vang, T., Cucca, F., and Mustelin, T. (2006) Semin. Immunol. 18, 207-213
11. Urbanek, R. A., Suchard, S. J., Steelman, G. B., Knappenberger, K. S., Sygowski, L. A., Veale, C. A., and Chapdelaine, M. J. (2001) J. Med. Chem. 44, 1777-1793
12. Beers, S. A., Malloy, E. A., Wu, W., Wachter, M. P., Gunnia, U., Cavender, D., Harris, C., Davis, J., Brosius, R., Pellegrino-Gensey, J. L., and Siekierka, J. (1997) Bioorg. Med. Chem. 5, 2203-2211
13. Andersen, J. N., Mortensen, O. H., Peters, G. H., Drake, P. G., Iversen, L. F., Olsen, O. H., Jansen, P. G., Andersen, H. S., Tonks, N. K., and Moller, N. P. (2001) Mol. Cell Biol. 21, 7117-7136
14. Alonso, Sasin, J., Bottini, N., Friedberg, I., Friedberg, I., Osterman, A., Godzik, A., Hunter, T., Dixon, J., and Mustelin, T. (2004) Cell 117, 699-711
15. Bottini, N., Musumeci, L., Alonso, A., Rahmouni, S., Nika, K., Rostamkhani, M., MacMurray, J., Meloni, G. F., Lucarelli, P., Pellecchia, M., Eisenbarth, G. S., Comings, D., and Mustelin, T. (2004) Nat. Genet. 36, 337-338
16. Begovich, A. B., Carlton, V. E., Honigberg, L. A., Schrodi, S. J., Chokkalingam, A. P., Alexander, H. C., Ardlie, K. G., Huang, Q., Smith, A. M., Spoerke, J. M., Conn, M. T., Chang, M., Chang, S. Y., Saiki, R. K., Catanese, J. J., Leong, D. U., Garcia, V. E., McAllister, L. B., Jeffery, D. A., Lee, A, T., Batliwalla, F., Remmers, E., Criswell, L. A., Seldin, M. F., Kastner, D. L., Amos, C. I., Sninsky, J. J., and Gregersen, P. K. (2004) Am. J. Hum. Genet. 75, 330-337
17. Lee, Y. H., Rho, Y. H., Choi, S. J., Ji, J. D., Song, G. G., Nath, S. K., and Harley, J. B. (2007) Rheumatology (Oxford) 46, 49-56
18. Carlton, V. E., Hu, X., Chokkalingam, A. P., Schrodi, S. J., Brandon, R., Alexander, H. C., Chang, M., Catanese, J. J., Leong, D. U., Ardlie, K. G., Kastner, D. L., Seldin, M. F., Criswell, L. A., Gregersen, P. K., Beasley, E., Thomson, G., Amos, C. I., and Begovich, A. B. (2005) Am. J. Hum. Genet. 77, 567-581
19. Gregersen, P. K., Lee, H. S., Batliwalla, F., and Begovich, A. B. (2006) Sem. Immunol. 18, 214-223
20. Todd, J. A., Walker, N. M., Cooper, J. D., Smyth, D. J., Downes, K., Plagnol, V., Bailey, R., Nejentsev, S., Field, S. F., Payne, F., Lowe, C. E., Szeszko, J. S., Hafler, J. P., Zeitels, L., Yang, J. H., Vella, A., Nutland, S., Stevens, H. E., Schuilenburg, H., Coleman, G., Maisuria, M., Meadows, W., Smink, L. J., Healy, B., Burren, O, S., Lam, A. A., Ovington, N. R., Allen, J., Adlem, E., Leung, H. T., Wallace, C., Howson, J. M., Guja, C., Ionescu-Tirgoviste, C., Simmonds, M. J., Heward, J. M., Gough, S. C., Dunger, D. B., Wicker, L. S., and Clayton, D. G. (2007) Nat. Genet. 39, 857-864
21. Hasegawa, K., Martin, F., Huang, G., Tumas, D., Diehl, L., and Chan, A. C. (2004) Science 303, 685-689
22. Cloutier, J. F., and Veillette, A. (1999) J Exp Med 189, 111-121
23. Alonso, A., Bottini, N., Bruckner, S., Rahmouni, S., Williams, S., Schoenberger, S. P., and Mustelin, T. (2004) J. Biol. Chem. 279, 4922-4928
24. Vang, T., Congia, M., Macis, M. D., Musumeci, L., Orru, V., Zavattari, P., Nika, K., Tautz, L., Tasken, K., Cucca, F., Mustelin, T., and Bottini, N. (2005) Nat. Gen. 37, 1317-1319
25. Booth, R. (Faculty of 1000 Biology, 15 Noy 2005 http://www.facultyof1000.com/article/16273109/evaluation)
26. Mustelin, T., Tautz, L., and Page, R. (2005) J. Mol. Biol. 354, 150-163
27. Kholod, N., and Mustelin, T. (2001) Biotechniques 31, 322-323, 326-328
28. Jones, G., Willett, P., Glen, R. C., Leach, A. R., and Taylor, R. (1997) J Mol Biol 267, 727-748
29. Goldgur, Y., Craigie, R., Cohen, G. H., Fujiwara, T., Yoshinaga, T., Fujishita, T., Sugimoto, H., Endo, T., Murai, H., and Davies, D. R. (1999) Proc Natl Acad Sci USA 96, 13040-13043
30. Clipstone, N. A., and Crabtree, G. R. (1992) Nature 357, 695-697
31. Weiss, A., and Stobo, J. D. (1984) J. Exp. Med. 160, 1284-1299
32. Overton, W. R. (1988) Cytometry 9, 619-626
33. Wu, J., Katrekar, A., Honigberg, L. A., Smith, A. M., Conn, M. T., Tang, J., Jeffery, D., Mortara, K., Sampang, J., Williams, S. R., Buggy, J., and Clark, J. M. (2006) J. Biol. Chem. 281, 11002-11010
34. Mustelin, T., Alonso, A., Bottini, N., Huynh, H., Rahmouni, S., Nika, K., Louis-dit-Sully, C., Tautz, L., Togo, S. H., Bruckner, S., Mena-Duran, A. V., and al-Khouri, A. M. (2004) Mol. Immunol. 41, 687-700
35. Arimura, Y., Vang, T., Tautz, L., Williams, S., and Mustelin, T. (2008) Mol. Immunol. 45, 3074-3084
36. Yu, X., Sun, J. P., He, Y., Guo, X., Liu, S., Zhou, B., Hudmon, A., and Zhang, Z. Y. (2007) Proc. Natl. Acad. Sci. 104, 19767-19772
37. Xie, Y., Liu, Y., Gong, G., Rinderspacher, A., Deng, S. X., Smith, D. H., Toebben, U., Tzilianos, E., Branden, L., Vidovic, D., Chung, C., Schurer, S., Tautz, L., and Landry, D. W. (2008) Bioorg. Med. Chem. Lett. 18, 2840-2844
38. Krishnamurthy, D., Karver, M. R., Fiorillo, E., Orru, V., Stanford, S. M., Bottini, N., and Barrios, A. M. (2008) J. Med. Chem. 51, 4790-4795
39. Burke, T. R., Jr., and Lee, K. (2003) Acc Chem Res 36, 426-433

What is claimed is:

1. A compound, or a pharmaceutically acceptable salt, solvate, or hydrate thereof,
wherein the compound comprises a general formula 1 from the general class 1, 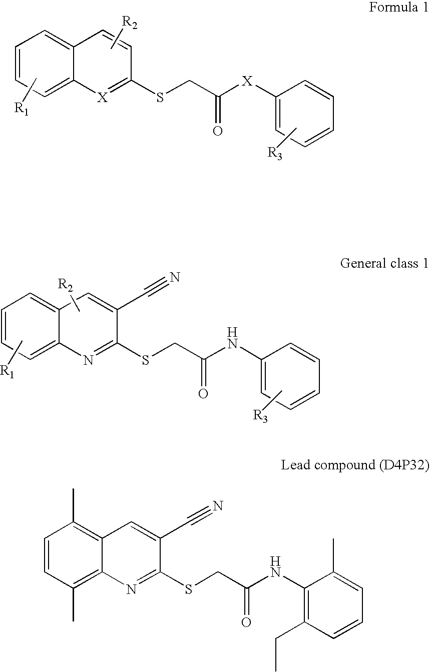
where each of R₁-R₃ is independently selected from the group consisting of a hydrogen atom, a halogen atom, a hydroxyl and a methoxyl group, and any other organic group containing any number of carbon atoms in a linear, branched, or cyclic structural format;
wherein each of X is a heteroatom such as N, O, S, and C; and
wherein formula 1 is derived from a lead compound DP432.

2. A compound, or a pharmaceutically acceptable salt, solvate, or hydrate thereof,
wherein the compound comprises a general formula 2 from the general class 2, 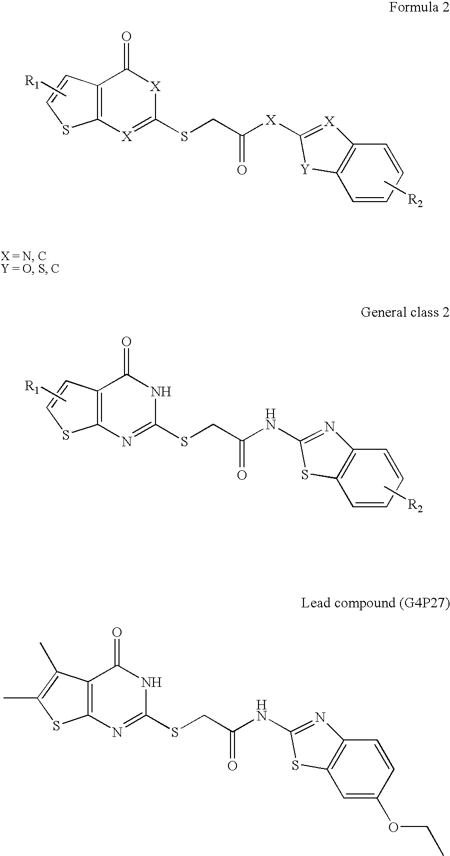
where each of R₁-R₂ is independently selected from the group consisting of a hydrogen atom, a halogen atom, a hydroxyl and a methoxyl group, and any other organic group containing any number of carbon atoms in a linear, branched, or cyclic structural format;
wherein each of X or Y is a heteroatom such as N, O, S, and C; and
wherein formula 2 is derived from a lead compound G4P27.

3. A compound, or a pharmaceutically acceptable salt, solvate, or hydrate thereof,
wherein the compound comprises a general formula 3 from the general class 3, 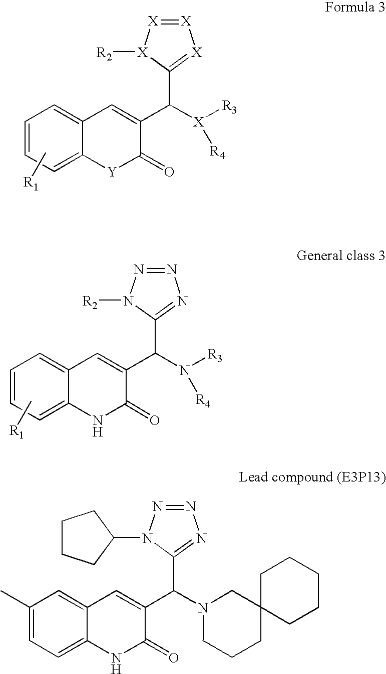
where each of R₁-R₄ is independently selected from the group consisting of a hydrogen atom, a halogen atom, a hydroxyl and a methoxyl group, and any other organic group containing any number of carbon atoms in a linear, branched, or cyclic structural format; and each of X or Y is a heteroatom such as N, O, S, and C; and
wherein formula 3 is derived from a lead compound E3P13.

4. A compound in accordance with claim 3, wherein the lead compound E3P13 comprises stereochemical mixtures or pure forms of E3P13 existing as either S or R configurations.

5. A compound, or a pharmaceutically acceptable salt, solvate, or hydrate thereof,
wherein the compound comprises a general formula 4 from the general class 4, 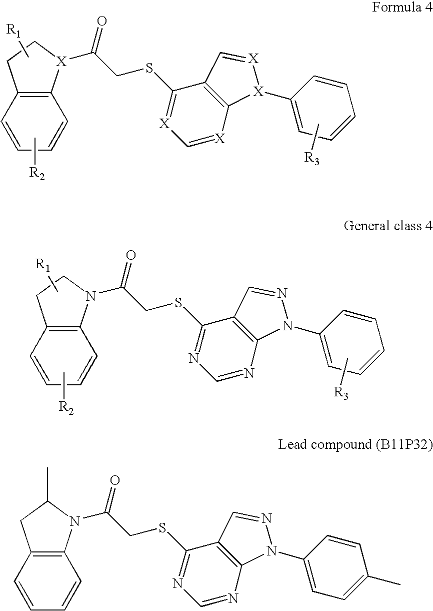
where each of R₁-R₃ is independently selected from the group consisting of a hydrogen atom, a halogen atom, a hydroxyl and a methoxyl group, and any other organic group containing any number of carbon atoms in a linear, branched, or cyclic structural format;
wherein each of X is a heteroatom such as N, O, S, and C; and
wherein formula 4 is derived from a lead compound B11P32.

6. A compound, or a pharmaceutically acceptable salt, solvate, or hydrate thereof,
wherein the compound comprises a general formula 5 from the general class 5, 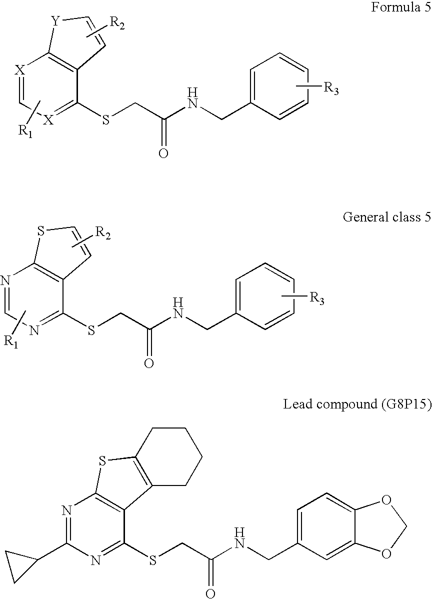
where each of R₁-R₃ is independently selected from the group consisting of a hydrogen atom, a halogen atom, a hydroxyl and a methoxyl group, and any other organic group containing any number of carbon atoms in a linear, branched, or cyclic structural format;
wherein each of X or Y is a heteroatom such as N, O, S, and C; and
wherein formula 5 is derived from a lead compound G8P15.
